(19) United States
(12) Patent Application Publication
(10) Pub. No.: US 2016/0030001 A1
(43) Pub. Date: Feb. 4, 2016

(54) Helmet Apparatus and System with Carotid Collar Means On-Boarded

(71) Applicants: Stuart Stein, Santa Ana, CA (US); Carlton R. Pennypacker, El Cerrito, CA (US)

(72) Inventors: Stuart Stein, Santa Ana, CA (US); Carlton R. Pennypacker, El Cerrito, CA (US)

(21) Appl. No.: 14/674,411

(22) Filed: Mar. 31, 2015

Related U.S. Application Data
(60) Provisional application No. 61/972,999, filed on Mar. 31, 2014.

(51) Int. Cl. A61B 8/00; A61B 19/00; A61B 8/12; A61B 8/06; A61B 8/02; A61B 8/08

(52) U.S. Cl. CPC A61B 8/4227; A61B 8/4488; A61B 8/02; A61B 8/488; A61B 8/4461; A61B 8/481; A61B 8/12; A61B 8/06; A61B 8/565; A61B 8/4416; A61B 19/2203; A61B 8/5207; A61B 8/5223; A61B 8/54; A61B 8/463; A61B 8/4411; A61B 8/486; A61B 8/5269; A61B 8/4263; A61B 8/483; A61B 8/5292; A61B 2019/5437

(57) ABSTRACT

Apparatus for helmeting with carotid collars works in conjunction with a transcranial Doppler, phased array photoacoustic device to transmit a first energy to a region of interest at an internal site of a subject to produce an image and blood flow velocities of a region of interest by outputting an optical excitation energy to said region of interest and heating said region, causing a transient thermoelastic expansion and produce a wideband ultrasonic emission. Systems integrate and register the signals for use in, for example, acute stroke care.

### RELATED APPLICATIONS

This application claims the benefit of and priority to U.S. provisional application No. 61/72,999 filed Mar. 31, 2014, which is incorporated by reference in its entirety. This application expressly incorporates by reference PCT application no. PCT/US2013/067713; U.S. patent application Ser. No. 14/070,264; U.S. provisional application No. 61/720,992; U.S. provisional application No. 61/794,618; and U.S. provisional application No. 61/833,802.

### FIELD OF THE INVENTION

The present invention relates to devices and methods to produce a more complete picture of a traumatic event in the brain, for example, a stroke, or cerebrovascular accident (CVA).

### BACKGROUND OF THE INVENTION

Strokes impact approximately 795,000 Americans each year. A stroke occurs when a vessel in the brain ruptures or is blocked by a blood clot. Although progress has been made in reducing stroke mortality, it is the fourth leading cause of death in the United States. Moreover, stroke is the leading cause of disability in the United States and the rest of the world. In fact, 20% of survivors still require institutional care after 3 months and 15% to 30% experience permanent disability. This life-changing event affects the patient's family members and caregivers. With an aging US population, the situation will only become more desperate.

Individuals afflicted with a stroke must receive immediate medical attention or risk suffering long term affects. However, many individuals suffering a stroke do not receive medical attention in time or are not diagnosed with a stroke. In some instances, patients are rushed to the closest hospital, but not the appropriate hospital equipped for treating a stroke patient. A hospital may be inappropriate because of inadequate diagnostic equipment, or lack of immediate access to required diagnostic and imaging testing. Also, the hospital may lack medical professionals, such as neurologists or interventional vascular specialists who are trained to give expert interpretation and necessary and warranted therapies. By the time the patient is diagnosed with a stroke, it may be discovered that the patient is at the wrong hospital and the potential for long term affects increases. In a stroke, 2 million nerve cells die per minute. Therefore, time is of the essence when diagnosing and treating stroke patients. It is best to start treatment within an hour of stroke onset. However, definitive stroke treatment can occur up to 4.5 hours after stroke onset with diminished efficacy of the treatment. In some instances, definitive therapy may not be available because the stroke has already occurred or is too large and cannot be reversed. If the patient arrives late, or is seen outside of the acceptable time window, or the patient has too many other medical risk factors to allow definitive therapy, then these factors may lead to complications, including brain hemorrhage. Also, screening of patients with stroke causing conditions is often not done. This can lead to a stroke, which may be preventable.

### SUMMARY OF THE INVENTION

The present invention provides a device for assessing a patient for a stroke, while in transport in ambulances, helicopters, or airplanes or in other diagnostic facilities. The device of the present invention allows for remote determination of parameters that indicate whether the patient is a possible stroke or a stroke risk patient. The results of the assessment allow for a patient to then be redirected to the nearest stroke treating hospital, thus saving valuable treatment time, allowing the preparation for and evaluation of the safest and most appropriate diagnosis and treatment.

The present invention relates to devices and methods for detecting oxygen levels, determining blood flow velocity, and imaging portions of the brain. The device collects these measurements from a patient. In some embodiments, the measurements are collected while the patient is in transport, and the measurements are sent to neurological and radiological team at a remote location, using advanced health information technology techniques. The neurological and radiological team determines whether a stroke has or is occurring, and can provide instructions to the transport team. Upon the patient's arrival at an appropriate hospital or emergency room, warranted and appropriate stroke diagnostics and treatments can begin immediately, thus saving valuable time. By arriving at the appropriate hospital, the correct therapy for the patients is chosen, thus maximizing the potential for better outcomes, including stroke reversal and reduced stroke severity, as well as reducing mortality. It is important to identify abnormalities or lack of blood flow in large neck or brain blood vessels, as these situations require the greatest time urgency, the greatest medical expertise for treatment, and also have the potential for the greatest brain damage.

Devices of the invention transmit energy to a region of interest in the patient head and neck regions. Energy can be delivered to cranial and carotid regions. In some embodiments, ultrasound transducers transmit and sense ultrasound waves to characterize a patient's brain and measure blood parameters. In some embodiments, ultrasound transducers transmit waves into a patient's head and neck region and the same transducers also sense the waves. The devices of the invention collect the ultrasonic waves and computers of the invention use the data to assess the patient, i.e. oxygen levels and blood flow velocity. In some embodiments, transducers are arranged on actuators. The actuators are able to move, rotate, or change the position of the transducers for aid in analysis and the collection of data.

Devices of the invention are configured to be placed on a patient's head and neck regions. The devices of the invention gather information about the blood flow and metabolism from the brain and the brain and neck vasculatures. For example, devices of the invention may be placed on the head and neck region of a patient in an ambulance, helicopter, or airplane to gather information about the blood flow in the head and neck region. The devices of the invention are able to send this data to a remote location. The device can also send this information to a doctor awaiting the arrival of the patient.

Thus, the devices and methods of the inventions provide remote, real-time, stroke diagnostics, as well as diagnostics applicable to other disorders that may mimic stroke or that may affect neck and brain blood flow, i.e. heart attack or diffuse infection (sepsis). The methods and devices of the invention are integrated into a telemedicine ecosystem, allowing for brain damage to be evaluated in real-time upon first-contact with patients, with a particular focus on narrowing or obstruction of large neck and brain blood vessels. Devices and methods of the invention capture neuro-vascular and metabolic information that is rapidly transmitted to a data and operations center for analysis by licensed neurologists, radiologists, and related professionals. Transmitting 3-D and 2D images of carotid and other neck arteries, and collecting blood flow velocities and other parameters on large or medium sized brain bloods vessels, and metabolic brain information during patient transport, the devices and methods of the invention allow professionals to render a diagnosis, inform EMT personnel, and alert the appropriate emergency room or stroke center to prepare for the pre-diagnosed patient. Thus, the present invention helps to differentiate among brain trauma, strokes, seizures, and intoxication, and hyper/hypoglycemic events, so that patients arrive at the right location, already diagnosed, saving valuable time and preventing the loss of up to two-million brain cells per minute in the event of a severe stroke.

In some embodiments, the devices of the invention deliver energy to a region of interest through a patient's head and neck region. In some embodiments energy may be delivered by a non-ionizing laser. Laser pulses are delivered into biological tissues. The energy is absorbed and converted into heat, causing transient thermoelastic expansion and thus wideband ultrasonic emission. The generated ultrasonic waves are detected by the ultrasonic transducers located on the device.

Thus, the devices and methods of the invention can be used to preventatively identify pre-stoke and stroke conditions that can lead to life-saving interventions-ranging from immediate removal of vascular obstructions to less invasive dietary and lifestyle changes. The present invention helps assure rapid treatment that saves lives, brain cells, expensive and time-consuming rehabilitation. In addition, pain, suffering, and other deleterious brain-related consequences are reduced.

### BRIEF DESCRIPTION OF THE DRAWINGS

FIG. 1 illustrates transcranial Doppler insonation of the cerebral circulation at a basic level.

FIG. 2 illustrates how Photoacoustic imaging works.

FIG. 3 schematically illustrates Novel Enhanced Apparatus with Helmet/Carotid Collar Means On-Boarded.

FIG. 4 further illustrates Novel Enhanced Apparatus with Helmet/Carotid Collar Means On-Boarded, according to the instant teachings.

FIG. 5 further illustrates Novel Enhanced Apparatus with Helmet/Carotid Collar Means On-Boarded, according to the instant teachings.

FIG. 6 further illustrates Novel Enhanced Apparatus with Helmet/Carotid Collar Means On-Boarded, according to the instant teachings.

FIG. 7 relates Novel Enhanced Apparatus with Helmet/Carotid Collar Means On-Boarded, to both the stroke ecosystem and data flow, according to the instant disclosures.

### DETAILED DESCRIPTION

The present invention provides methods and devices for diagnosing strokes in patients acutely in prehospital environments, and and potentially, preventatively. The devices and methods also identify and define vessels and metabolic abnormalities in patients in preventative settings that are at stroke risk. There are two types of strokes: hemorrhagic or ischemic. An ischemic stroke occurs as a result of an obstruction within a blood vessel supplying blood to the brain. It accounts for 87 percent of all stroke cases. A hemorrhagic stroke occurs when a blood vessel ruptures and spills blood into brain tissue. The treatment approaches are different for stroke without hemorrhage versus stroke with hemorrhage. For example, stroke patients without hemorrhage may require vessel opening therapies with intravenous thombolytics or intraarterial clot busters or catheter-based interventional clot removal. The latter treatments are dangerous and not warranted for hemorrhagic stroke.

Treating an acute stroke patient is time sensitive. However, many patients do not receive the required medical attention in time. The present invention provides devices and methods for early detection and diagnosis of stroke patients to afford the possibility for appropriate and safe treatment modalities acutely and to limit the occurrence of secondary complications, including brain hemorrhage. Further, this device provides a simple means for application to collect physiological data without significant technical expertise or time commitment by emergency technical providers.

An ischemic stroke is the result of neuronal death due to lack of oxygen, a deficit that produces focal brain injury. This event is accompanied by tissue changes consistent with an infarction that can be identified with neuroimaging of the brain. Strokes are usually accompanied by symptoms, but they also may occur without producing clinical findings and be considered clinically silent.

Both acute and chronic conditions may result in cerebral ischemia or stroke. Acute events that can lead to stroke include cardiac arrest, drowning, strangulation, asphyxiation, choking, carbon monoxide poisoning, and closed head injury. More commonly, the etiology of stroke is related to chronic medical conditions including large artery atherosclerosis, atrial fibrillation, left ventricular dysfunction, mechanical cardiac valves, diabetes, hypertension and hyperlipidemia. Thus, a patient may be recognized as suffering from cardiac arrest, but the presence of a stroke may go undetected. Regardless of the cause, prompt recognition of symptoms and urgent medical attention are necessary for evaluation and institution of clinically warranted thrombolytic or clot busting therapy through the veins or catheter and stent retriever related intra-arterial clot busting therapy or clot removal to be considered and provided.

Time is of the essence for beginning therapy and performing suitable evaluations. Clinical imaging and other testing may be performed during that time. Because time is so critical for performing neurological examination, imaging and other testing needs to occur during a critical time window. This has prompted increased education and awareness campaigns for the public and emergency services providers about the signs and symptoms of stroke. This has also established national protocols for acute stroke diagnosis and treatment to be adopted at increasing number of United States hospitals and their emergency departments. The current patent application is built on novel enhancement of existing established National protocols. The arrival of a stroke patient in the emergency room (ER) must be viewed as a true emergency, and the patient should receive the highest priority. On arrival to the ER, identification of the patient with a potential stroke should prompt the collection of several important data points: time the patient was last known to be neurologically normal; detailed neurological exam, including the use of National Institutes of Health Stroke Scale (NIHSS); determination of the neurological diagnosis and the severity of the neurological dysfunction; time known to last be neurologically normal; serum glucose level; general metabolic screening; blood count and blood clotting status screening; recent and remote medical and neurological history, with particular attention to diabetes, hypertension, recent surgery or head injury; prior bleeds in brain and other tissues, and epilepsy; current medications, allergies, and baseline CT scan of head for stroke, hemorrhage or other condition. Potential stroke and determination of risk and eligibility or clot buster or intervention brain or neck artery therapy are derived from this evaluation. Rapid, safe and appropriate therapy for specific patients is fostered by rapid assessment as documented above.

Recently, since neurological evaluation with stroke specialists may not be uniformly available rapidly or geographically, stroke telemedicine using tele-neurologists at remote locations with special mobile audio video equipment in the Emergency Department or other settings can provide review of all relevant data, neurological examination, and CT scan review, while advising Emergency physicians about appropriate and safe therapies. This is helpful within the time window and similar in concept to the rapid determination of physiological measures pre-hospital in the current application. In the current embodiment, physiological measurements with carotid and transcranial Dopplers are provided in real time by telemedicine to operating centers staffed by expert teleradiologists and teleneurologists that also provide real time analysis to allow for stroke patient transport to appropriate stroke centers that are prepared to provide rapid diagnostics and appropriate and warranted treatment.

Certain brain and neck imaging modalities are essential for the rapid evaluation of stroke. At the time of stroke, mini-strokes, suspected strokes, or transient ischemic attacks (TIAs), a CT scan of brain is initially mandated and done first to look for bleeding or brain hemorrhage, stroke presence and size, or other diagnosis. When normal, treatment decisions have to be based on neurological examination and other criteria documented above. When hemorrhage is present, the patient follows a different but rapid treatment pathway. An embodiment within the current application provides a means to distinguish stroke with hemorrhage from stroke without hemorrhage, using phased array, carotid and/or transcranial Doppler and photoacoustic spectroscopy.

CT scan of the brain is routinely available in most hospitals, emergently whether stroke ready, or primary or comprehensive stroke centers. Expert reading of the data may or may not be available or available within the required time frame. Magnetic resonance imaging (MRI) of the brain (intracranial) is more sensitive and specific for stroke and for therapy risk assessment for stroke than CT scan for stroke presence and severity and therapy risk evaluation, but in the majority of hospital and emergency settings, MRI is not physically available or with rapid expert interpretation rapidly, i.e. within 15-20 minutes and is expensive. The embodiments outlined herein can be used alone or in combination with CT and when MRI is available, to provide a sensitive and specific means to decide on appropriate rapid therapies for acute stroke and help to delimit risk. The current embodiments will provide an alternative imaging method for distinction of stroke with brain hemorrhage from stroke without hemorrhage that would affect the type of treatment, while also saving time.

As embodied in this application, 2D and 3D carotid doppler and transcranial doppler employed in pre-hospital evaluation can replace vessel imaging in multiple circumstances and for many reasons; the embodiments used to look for blood vessel abnormalities, including stenosis and obstruction of the main brain arteries, including the middle cerebral arteries and basilar artery, and neck arteries, carotids, as a basis for stroke and for specific intravenous clot buster therapy and intra-arterial clot buster or catheter based clot retrieval therapy.

### Brain Perfusion and Oxygenation in Normal and Acute Stroke

The embodiments in the current application with carotid and/or transcranial Doppler, phased array, and photoacoustic spectroscopy, discussed in detail below, provide a means to determine brain perfusion in stroke in a pre-hospital environment and replace MRI and CT perfusion scans, that may provide useful information in acute stroke care; MRI and CT perfusion have limitations. In the hospital and emergency department, stroke evaluation setting, magnetic resonance perfusion (MR perfusion) or CT perfusion may be employed to further define a stroke with irreversible dead brain tissue, i.e. completed stroke and without sufficient oxygenation versus injured but reversible components of the potential stroke. The latter is called the penumbra. These penumbras would have oxygen present to varying degrees and varying tissue viability. These penumbra can progress to stroke or be reversed completely or partially. Penumbra are the targets for early stroke therapy.

The penumbra and its characteristics may help guide safe and appropriate therapeutic decisions, including limiting brain hemorrhage secondary to intravenous or intraarterial clot buster or intra-arterial clot buster or clot retrieval therapies. MR perfusion depends on water molecule behavior in stroke and not on true measures of tissue oxygenation. However, the validity of MR and CT perfusion, the advantages of MR versus CT perfusion, the time and availability for performance (except at generally the small number of comprehensive stroke centers and some primary stroke centers) and for interpretation, limit the current utility of these potentially useful techniques. Current measures of this and above and beyond CT perfusion and MR perfusion include xenon-CT, single photon emission computed tomography (SPECT), positron emission tomography (PET), but these are not available in acute settings, may be time consuming for performance, and interpretation, and are only available in limited selected locations. Invasive means for cerebral perfusion using thermal diffusion probes are not relevant for acute stroke.

In acute stroke, brain tissue oxygenation determination is important and cerebral perfusion reflects both oxygenation and necessary metabolite delivery. In the current embodiment, this is evaluated and supplants unavailable and existing protocols with limitations as detailed above. The embodiments presented in this application provide a means to look at brain oxygenation as well as compliment the analysis of treatment efficacy and patient improvement by working in concert with established interventional treatments for acute stroke. Determination of brain oxygenation or brain tissue oxygen tension has been and is done with optical luminescent and polarigraphic methods. Both techniques require placement of probe directly within brain tissue which is not applicable in acute pre-hospital or emergency department stroke evaluation or preventative settings for stroke.

### 2D and 3D Carotid Doppler, Transcranial Doppler and Interventional Angiography for Acute Stroke

The embodiments presented in this application provide a means to go directly to interventional therapy, which initially involves brain and neck catherter-based angiography. Brain and neck vessel angiography, which visualizes brain and neck artery anatomy precisely with contrast, is warranted in those patients appropriately selected to get intra-arterial clot buster or clot retrieval or in surgical procedures to physical remove clot from neck (carotid) arteries. These techniques are the gold standard in brain and neck vessel definition, with particular relevance to obstruction and collateral blood flow. These techniques provide the platform for delivery of clot buster to blocked arteries or unblocking of those arteries with intra-artery clot buster or clot retrieval with or without stenting. These techniques also provide information after the obstruction clearing attempts at successful opening of the blocked or obstructed vessels. These angiography techniques only provide an anatomical picture but no information on tissue efficacy of potentially damaged tissue before, during, or after the procedure. Oxygenation efficacy of damaged and surrounding tissue at these times cannot be assessed with angiography but could be intravascularly assessed with carotid and/or transcranial Doppler, phased array, and photoaccoustic spectroscopy.

Additionally with our embodiments, real time analysis of blood flow velocity and flow direction and other neck and brain blood flow measures is not available pre-hospital or in the Emergency Department. These can be provided with carotid Doppler and transcranial Doppler and phased array ultrasound. Also, known techniques, except transcranial Doppler, are unable to detect and characterize brain or neck artery emboli, define vessel plaque characteristics, and measure vessel-wall thickness (carotid Doppler with intimal thickness). Additionally, no current CT, MRI, or cerebral angiography can look at brain tissue oxygenation as discussed above. Additionally, all CT and MR techniques cannot be done pre-hospital and these techniques require time, which may be at the expense of potentially saving brain in acute stroke.

### Brain and Neck Ultrasound Examination

Transcranial Doppler and Carotid Doppler provide for real time analysis of brain and neck blood flow that compliment anatomical representations of brain and neck arterial anatomical imaging, i.e. CT and MR angiography of head and neck. A piezoelectric crystal emits ultrasound pulses and listens for reflected echos (sound waves). The reflected echos may provide time of flight, intensity, or frequency data of the reflected versus the transmitted wave. Velocity of blood flow is based on the calculated frequency shift of reflected waves.

Both transcranial and carotid Doppler are performed in a standard sequence that involves placement upon sites to insonate the vessels, listening for the sound of blood flow that may reflect on normal flow or obstructed flow, and determination of anatomical vessel characteristics (carotid Doppler), spectral analysis with blood flow velocity and pulse wave determination (carotid and transcranial Doppler), adventitial embolic signals (transcranial Doppler), power m mode (transcranial Doppler) and comparison of anatomical and blood flow velocity and wave anatomy with known, established, and normal standards to determine normal versus abnormal, including determination of abnormal vessels with degree of stenosis and obstruction. Low or elevated blood flow may reflect on local pathology of the neck or brain blood vessels or the efficacy of blood flow from the heart, i.e. cardiogenic shock, cardiac valve disorders, or sepsis. Rapid and real time transcranial Doppler and carotid Doppler can identify critical stenosis or obstruction in specific neck and brain blood vessels that will provide information for correct hospital transport, hospital preparation for stroke intervention, appropriate treatment selection, and time savings to save brain cells.

### Carotid Doppler

Vascular duplex ultrasound of the carotid Doppler involves 2 ultrasound components, B-mode Gray Scale (2-D imaging) and Doppler imaging including flow measurement, color Doppler and spectral Doppler with blood flow velocity measurement. In the current embodiment, carotid Doppler will include the above elements and will be recorded with a previously validated (NASA Space Simulator) carotid Doppler system and transducers affixed to the bilateral carotid arteries. The transducer will be a standard 4 cm or larger convex as opposed to linear transducer. A single sampling point will be used as opposed to multiple sampling points for proximal, middle, and distal carotid arteries. Raw imaging data will be sent wirelessly to the data/operations center, processed there, and analyzed similar to the transcranial Doppler ultrasound. The carotid Doppler probes will be used to evaluate the carotid arteries and the neck vertebral arteries. The carotid Doppler probe will be incorporated into the neck portion of the helmet (figures). The internal carotid artery is particularly relevant for stroke.

B mode or gray scale imaging can look at the carotid artery and associated anatomical vessel and other structures in transverse or longitudinal plane. B mode is useful for defining the internal carotid artery wall and characterizing, localizing and defining extent and size of low or high echo structures, including atherosclerotic plaque, that may be obstructing the vessel, i.e. internal carotid artery. Plaque usually results from aging change and pieces of plaque may dissociate and lead to emboli sent distally. Carotid artery tearing, plaque obstruction, or emboli from plaque or carotid artery spasm, bleeding into the carotid wall, can all lead to stroke. Information related to these causes can be derived from B mode imaging.

Complimentary to B mode imaging, color flow Doppler can reveal blood flow direction and mean velocity of flow and is very useful for imaging stenosis or obstruction and the site within the vessel. At various levels of the carotid artery, the peak and mean flow velocities, resistance, and actual arterial wave on spectral imaging provides quantitative numbers for determination of obstruction and stenosis of the internal carotid artery. All elements of the carotid doppler examination as well as information on the vertebral arteries in the neck can be rapidly accessed and used for rapid evaluation of stroke, its cause, and potential intervention. Other arteries can be assessed in the neck as part of the internal carotid artery examination.

### 3D Carotid Doppler

In another embodiment, multiple 2D carotid images can be rapidly obtained through the current carotid ultrasound device and processed and reconstructed into a 3D or 3 dimensional image of the carotid artery. The latter incorporates B mode and color flow Doppler. Rapid identification of stenosis and obstruction can be demonstrated with combined individual 2D internal carotid Doppler and separate 3D carotid Doppler.

### Transcranial Doppler

The devices and methods of the invention employ Transcranial Doppler (TCD). TCD is a test that measures the velocity of blood flow through the brain's blood vessels, usually the mean blood flow velocity. Blood flow velocity is recorded by emitting a high-pitched sound wave from the ultrasound probe, which then bounces off of various materials to be measured by the same probe. A specific frequency is used (usually close to 2 MHz), and the speed of the blood in relation to the probe causes a phase shift, wherein the frequency is increased or decreased. This frequency change directly correlates with the speed of the blood, which is then recorded electronically for later analysis. Normally a range of depths and angles must be measured to ascertain the correct velocities. For transcranial Doppler, the site of insonation determines the potential vessels to be sampled, i.e. pre-temporal for example is for middle cerebral arteries or anterior cerebral arteries. This technique is an indirect measure and depth of insonation by power m mode is directly related to the position on a specific artery.

Because the bones of the skull block the transmission of ultrasound, regions with thinner walls insonation windows must be used for analyzing. For this reason, recording is performed in the temporal region above the cheekbone/zygomatic arch, through the eyes, below the jaw, and from the back of the head. Patient age, gender, race and other factors affect bone thickness, making some examinations more difficult or even impossible. Most can still be performed to obtain acceptable responses, sometimes requiring using alternate sites from which to view the vessels.

Transcranial Doppler is a real time technique that is sensitive and specific for blood flow velocity in multiple medium and large blood vessels of the brain over a broad range of velocities, able to determine brain blood vessel resistance, useful in determining collateral flow presence and efficacy and cerebral atherosclerosis, able to compare blood flow in blood vessels in comparison from one side of the brain to the other, is the only technique available for brain emboli detection, and can reliably predict vessel obstruction. Transcranial Doppler images can give specific artery and within artery information on mean flow velocity, flow direction, and obstruction and stenosis. Wave analysis on spectral flow is also useful in defining site of stenosis or obstruction as well as efficacy of blood flow. Transcranial Doppler analysis follows a sequential analysis of the ophthalmic vessels, the vessels in the anterior circulation, noted pre-temporally, and the posterior circulation at the back of the head, with continuous listening for bruits and atherosclerosis and also emboli followed by prolonged emboli detection. Specific abnormalities in the waveform and also specific velocities may be associated with obstruction and stenosis when compared to normal age related standards for specific vessels.

In this embodiment, eye patch transcranial doppler probes will be applied to the eyelids to sample the ophthalmic arteries bilaterally and transcranial probes will be used in the pre-temporal region to evaluate the middle and anterior cerebral arteries and other arteries bilaterally and in the back of the head to evaluate the basilar artery and vertebral arteries and other arteries (See prior patents). The transcranial Doppler probes will be incorporated in the pre-temporal region and in the back of head (suboccipitally) into the helmet (figures). Transcranial Doppler mean blood flow velocity in major cerebral arteries represents an indirect assessment of cerebral perfusion. Changes in cerebral blood flow can be inferred from changes in blood flow velocity; however, there are limitations in that a constant vessel diameter and specific angle of insonation are assumed. Transcranial Doppler can not measure perfusion abnormalities at the microcirculatory level but large vessel territory perfusion abnormalities are relevant in stroke definition and determination for intervention. Operator expertise has limited transcranial Doppler but is obviated by the embodiments of the helmet and probe design.

### Phased Array

Phased Array Ultrasound enables the use of multiple transducers to be pulsed and readout independently. Having an array of such devices enables beam steering, beam forming, and higher resolution imaging upon return of the reflected/scattered ultrasound. Due to the larger receiving aperture, the beam can be electronically steered, and then read back for that part of space interrogated by the smaller beam size enabled by the phased array beam-forming algorithms. Such devices are used in Medical Imaging and in many industrial applications. Typically, because of the much higher resolution afforded by MRI and CT scanning devices, phase array ultrasound has not been used in the brain. However, when larger structures are imaged, such as major vasculature, and superb resolution is not desired, phased array ultrasound is adequate. In particular, phased array ultrasound can fit into a small box, of size 10″×10″×3″, and be part of an ambulances or Emergency Department or other medical settings, equipment, as compared to the room-size MRI's and CT scanning systems in common use. Phased array has been used to look at brain blood flow velocities, similar to transcranial Doppler and the probes could be placed in similar positions to transcranial Doppler probes.

In the current embodiment, phased array probes could replace transcranial Doppler probes. This would provide beam steering capacities that may increase the procurement of brain vessel data. In the current embodiment, in addition to external use within the helmet, a phased array probe or transcranial Doppler probe would combined with an optoacoustic or photoacoustic probe to provide physiological vessel flow data, reflective of stenosis or obstruction, and oxygenation information on contiguous brain tissue that is supplied by these vessels (See below). It should be appreciated that probes and transducers are synomous and can be used interchangeable in the application, and the probes of the invention and could be carotid probes, transcranial probes, phased array or photoacoustic spectroscopy probes.

### Photoacoustic Spectroscopy

The devices and methods of the invention employ photoacoustic spectroscopy as part of the evaluation of oxygen and oxygenation externally in some embodiments. For example, probes for this would be added to the existing head parts of the helmet (not shown). Further, as part of this embodiment, a photoacoustic head would be part of the transcranial Doppler and phased array multihead probes that would be used in intravascular evaluation in connection with cerebral angiography and interventional catheter based intrarterial therapy with clot buster or clot removal/stenting. Photoacoustic spectroscopy is the measurement of the effect of absorbed electromagnetic energy (particularly of light) on matter by means of acoustic detection. The discovery of the photoacoustic effect dates to 1880 when Alexander Graham Bell showed that thin discs emitted sound when exposed to a beam of sunlight that was rapidly interrupted with a rotating slotted disk. The absorbed energy from the light is transformed into kinetic energy of the sample by energy exchange processes. This results in local heating and thus a pressure wave or sound. Later Bell showed that materials exposed to the non-visible portions of the solar spectrum (i.e., the infrared and the ultraviolet) can also produce sounds.

Photoacoustic imaging is based on the photoacoustic effect. In photoacoustic imaging, non-ionizing laser pulses are delivered into biological tissues (when radio frequency pulses are used, the technology is referred to as thermoaccoustic imaging).

Some of the delivered energy will be absorbed and converted into heat, leading to transient thermoelastic expansion and thus wideband (e.g. MHz) ultrasonic emission. The generated ultrasonic waves are then detected by ultrasonic transducers. Computer systems of the invention convert these waves into images. It is known that optical absorption is closely associated with physiological properties, such as hemoglobin concentration and oxygen saturation.

Hemoglobin (Hb or Hgb) is the iron-containing oxygen-transport metalloprotein in the red blood cells of most vertebrates. Hemoglobin in the blood carries oxygen from the respiratory organs (lungs or gills) to the rest of the body (i.e. the tissues) where it releases the oxygen to burn nutrients to provide energy to power the functions of the organism, and collects the resultant carbon dioxide to bring it back to the respiratory organs to be dispensed from the organism.

In general, hemoglobin can be saturated with oxygen molecules (oxyhemoglobin), or desaturated with oxygen molecules (deoxyhemoglobin). Oxyhemoglobin is formed during physiological respiration when oxygen binds to the heme component of the protein hemoglobin in red blood cells. This process occurs in the pulmonary capillaries adjacent to the alveoli of the lungs. The oxygen then travels through the blood stream to be dropped off at cells where it is utilized as a terminal electron acceptor in the production of ATP by the process of oxidative phosphorylation. It does not, however, help to counteract a decrease in blood pH. Ventilation, or breathing, may reverse this condition by removal of carbon dioxide, thus causing a shift up in pH. In this embodiment both as part of the external headset apparatus or the brain intra-arterial set of probes, photoacoustic spectroscopy would be used to evaluate oxygenation, tissue efficacy, and as part of the determination of cerebral perfusion in combination with transcranial doppler and phased array ultrasound and special fluorescent intravascular injection.

### Vasculature and Perfusion Measurement

In some embodiments, the devices and methods of the invention employ perfusion.

Perfusion is the process of delivery of blood to a capillary bed in the biological tissue. Vasculature and perfusion measurements in the brain perfusion (more correctly transit times) can be estimated with contrast-enhanced computed tomography or MR angiography. To get a better representation of the blood flow in the brain, a dye is injected into the patient to enhance visualization of the suspect area. Cerebral perfusion measurements are based on quantitative measures of cerebral blood flow, mean transit time (MTT), or time to peak flow (TTP and cerebral blood volume (CBV). In some embodiments, brain perfusion in specific regions of potential completed stroke and penumbral regions with still preserved function will involve transcranial doppler, phased array, photoacoustic spectroscopy, and ICN dye.

Tissue plasminogen activator (tPA) or clot buster is used in diseases that feature blood clots, such as stroke, pulmonary embolism, myocardial infarction, and stroke, in a medical treatment called thrombolysis. To be most effective in ischemic stroke, tPA must be administered as early as possible after the onset of symptoms. Protocol guidelines require its use intravenously within the first three hours of the event (in some cases up to 4.5 hours), after which its detriments may outweigh its benefits. tPA can either be administered systemically or administered through an arterial catheter directly to the site of occlusion in the case of peripheral arterial thrombi and thrombi in the proximal deep veins of the leg. In some embodiments, the methods and devices include introducing iPA intravenously or intra-arterially into a patient after assessing a patient for a stroke and evaluating for potential risk of this therapy in each specific patient situation.

In an embodiment of the present invention, a method of configuring a transcranial Doppler photoacoustic device to transmit a first energy to a region of interest at an internal site of a subject is disclosed (the entire inside of the skull is illuminated, and produces sound waves, proportional to the absorption of incident light). The method comprises the steps outputting optical excitation energy to said region of interest and heating said region, causing a transient thermoelastic expansion and producing a wideband ultrasonic emission. A phased-array transducer system records the ultrasonic waves. Computer systems of the invention convert the waves into images. Because all of the transducers record simultaneously, the device can image the whole brain area simultaneously.

By, providing at least one, or a plurality of one or two dimensional detectors, the detectors receive wideband ultrasonic emission. An oxygen level is computed of said region of interest from said wideband ultrasonic emission. Then, an array of ultrasound transducer elements output a beam pattern from said array of ultrasound transducer elements to insonate a region of interest at an internal site in a body, where the beam output pattern is sufficiently large to comprise a multi-beam pattern. Multiple receiver elements insonate over a substantially simultaneous period by directing energy produced by said array of ultrasound transducer elements into said region of interest in said body, and adjusting an amplitude of energy output by said array of transducers to cause the beam pattern output to have a generally flat upper pattern and nulls in a grating lobe region. This would be performed by the user with the device and associated software.

Then a propagation time delay is introduced and the beam pattern output from said array of ultrasound transducer elements, wherein the propagation delay increases as a distance increases from a central output area of said array of ultrasound transducer elements produces an image of said internal site. In addition, in software during reconstruction, phase shifts can be selectively added to all of the signals so that the reconstructed beam scans the whole brain cavity.

The photoacoustic technology deployed in the devices of the invention uses an unfocused detector to acquire the photoacoustic signals and the image is reconstructed by inversely solving the photoacoustic equations. Alternatively, the transcranial Doppler photoacoustic device of this embodiment may use a spherically focused detector with 2D and 3D point-by-point scanning and would require a reconstruction algorithm. Thermoelastic expansion of the blood vessel wall depends on the oxyhemoglobin/deoxyhemoglobin ratio. In order to obtain precise mapping of the area of interest, the Doppler ultrasound functionality of the device is utilized to provide an image to the user.

Aspects of the invention allow the device to be placed on a patient in an ambulance, or at a remote location. An advantage to the present invention is that it allows for identifying a potential stroke by providing brain insight data to the stroke team in advance of the patient's arrival. The devices and methods provide a depiction of the middle cerebral arteries and carotid arteries and then basilar Artery. Tomography of oxygenation in three regions of middle cerebral artery territory and two regions of basilar artery can also be performed with the invention.

In other embodiments, a dye will be given to visualize the brain vasculature and a perfusion measurement can be made in various regions of the brain along with the transcranial Doppler and the photoacoustic screening.

The photoacoustic technology deployed in this device uses an unfocused detector to acquire the photoacoustic signals and the image is reconstructed by inversely solving the photoacoustic equations. Alternatively, the transcranial Doppler photoacoustic device of this embodiment may use a spherically focused detector with 2D and 3D point-by-point scanning and would require a reconstruction algorithm, that operates in near real-time or after data acquisition is complete. Thermoelastic expansion of the blood vessel wall depends on the oxyhemoglobin/deoxyhemoglobin ratio. In order to obtain precise mapping of the area of interest, the Doppler ultrasound functionality of the devise is utilized to provide an image to the user.

An exemplary embodiment of the present invention provides an efficient method and device for configuring a laser-induced photoacoustic tomography (PAT) device (photoacoustic spectroscopy). The advantage of PAT over pure optical imaging is that it retains intrinsic optical contrast characteristics while taking advantage of the diffraction-limited high spatial resolution of ultrasound. This embodiment will also allow for imaging hyperoxia- and hypoxia-induced cerebral hemodynamic changes. The PAT technology would show oxygenation levels and the phased array Doppler would present blood flow. This embodiment employs an algorithm of using velocities and blood distribution and oxygen level to simultaneously to determine what is going on with neuronal respiration. This algorithm will determine the 12 types of strokes, as treatment is different in a hemorrhagic stroke or an emboli-induced stroke, in that the blood distribution and velocities are far different in each type.

In an additional embodiment, a microwave-based thermoaccoustic tomography (TAT) device would be used to image deeply seated lesions and objects in biological tissues and the phased array Doppler or single receiver Doppler would present blood flow. Because malignant tissue absorbs microwaves more strongly than benign tissue, cancers can be imaged with good spatial resolution and contrast.

In still yet another embodiment the phased array Doppler would present blood flow using multiple wavelength photoacoustic measurements. Oxoborinic acid (Hb0₂) is the dominant absorbing compounds in biological tissues in the visible spectral range, multiple wavelength photoacoustic measurements can be used to reveal the relative concentration of these two chromophores (the part of a molecule responsible for its color). Thus, the relative total concentration of hemoglobin (HbT) and the hemoglobin oxygen saturation (s0₂) can be derived. Therefore, cerebral hemodynamic changes associated with brain function can be successfully detected with PAT. For example, under a hyperoxia status, the averaged s0₂ level, in the areas of imaged cortical venous vessels of brain is higher than that under the normoxia status.

In an additional embodiment, the devices and methods can be placed on the skull at multiple points without concern for skull bone interference.

Additional embodiments would allow a user to track blood flow and obtain real time oxygenation levels to map out circulatory patterns.

In yet another embodiment, certain compounds in vascular walls could be excited by either phased array Doppler or the PAT. This would allow the user to analyze the atheroma (plaque) on the linings of certain compounds on vasculature walls.

In an additional embodiment, the ultrasonic transducers are configured in different patterns to aid in the reception of the photoacoustic signal, for example, the transducers can be set up in an 8 by 8 array.

In still yet another embodiment, an algorithm deployed as software, firmware or hardware will produce data which can utilized to produce an image of the biological tissue. In an another embodiment, a tunable laser would be utilized for subtraction and comparison differential imaging to see emboli, say in the carotid artery, or subclavian artery, which are not underneath the skull or any additional areas of interest.

In still yet another embodiment, different frequencies of light of the laser would excite vascular wall, gaseous emboli, and fatty emboli, in superficial or deeper vasculature, both in the skull or the general circulation, to determine probability or likelihood of stroke or other vascular disorder.

The devices and methods of the present invention, transcranial doppler, detect emboli in the brain. Emboli may be gaseous or particulate. Examples of emboli include calcium, fat, platelets, red blood cells, clots, or other substances that travel through the bloodstream and lodges in a blood vessel. A stroke or transient ischemic attacks (TIA) involve brain tissue damage that results from the obliteration of blood flow with reduced oxygen delivery through specific extracranial vessels, i.e. carotid arteries, cervical vertebral arteries, or intracranial vessels, i.e. middle cerebral arteries, posterior cerebral arteries due to atherosclerotic vessel change, emboli, or a combination of both. The size of these embolic components is approximately 50 microns for particulate or solid emboli and 1-10 microns for gaseous emboli. Particulate emboli may have a more important role in stroke or TIA causation, as compared to gas emboli; this underlies a need for detection and differentiation of particulate versus gas emboli.

Cerebral emboli may be associated with cardiac, aorta, neck and intracranial vessel disease, as well as coagulation disorders and neck and during diagnostic and surgical procedures on the heart and the carotid arteries. Cerebral embolism can be a dynamic process episodic, persistent, symptomatic, asymptomatic, and may, but, not in all cases, predispose to stroke or TIA, influenced to some degree by composition and size; the latter embolic stroke, which is influenced by the vessel and its diameter to which the embolus goes.

In another embodiment, a method for allowing an ambulance crew or EMTs (Emergency Medical Technicians) to evaluate a stroke out in the field or on the ambulance's way to the E.R., (it should be appreciated that ER, emergency room) the ambulance would be outfitted with the present invention which would send valuable telemetry to the E.R. ahead of the patient's arrival. The steps would include dispatching an ambulance and EMT to the patient. A Transcranial Doppler of Bilateral Middle Cerebral Arteries and Carotid Arteries and then Basilar Artery would be performed (These are the large arteries that can cause the most severe stroke and that would be amenable to intravenous or intraarterial therapy). Then a Photoacoustic Tomography (photoacoustic spectroscopy) of oxygenation in three regions of middle cerebral artery territory and two regions of basilar artery would be performed.

The data would be sent to an operations center where it would be rapidly processed; the processed data would be rapidly evaluated by experienced neurologists and radiologists at the operations center. The analysis of this data would be provided to the ambulance, providers at the stroke center or hospital or emergency room, including neurologists and radiologists. A decision would then be made rapidly as to the hospital destination for the ambulance that would maximize care quality, specific imaging and expert availability, and reduce time to evaluation and therapy. Further, preparation of imaging needs, clot buster mixing, other protocol requirements for diagnostics, and preparation of the angiography suite and personnel for rapid intra-arterial clot buster or clot retrieval would be promoted by this plan. All of this would be done prior to the patient's arrival at the hospital and emergency department. The current embodiment and its associated stroke ecosystem would foster a logistical operation that would reduce time and maximize potential appropriate therapy, reduce risk, and improve patient prognosis.

In an embodiment of the above, a dye is given with visualization of brain vasculature and perfusion measurement made in same regions as with the original Photoacoustic Tomography. In another embodiment of the present invention, a method for determining the correct therapeutic procedure, includes determining if major obstruction in carotid arteries, MCAs, or basilar along with large scale view of all vessels performed, then determining if there are regional oxygenation reductions in these territories. Then to determine if diffusion (from flow)/perfusion mismatch in brain sub regions and finally identify therapeutic alternative including IV or IA tPA or intra-arterial clot removal/retrieval.

In another embodiment of the present invention, a transcranial Doppler or phased array transcranial Doppler are deployed to determine cerebral blood flow velocity. A photoacoustic tomography is performed to determine brain oxygenation.

In combination with transcranial doppler, phased array, and photoacoustic spectroscopy, indocyanine Green dye is injected to determine cerebral perfusion and mean transit time of the blood. This permits the care provider to determine if the patient has an irreversible stroke or a reversible vascular injured brain.

In another embodiment of the present system and method, an early cancer, particularly prostate cancer detection device is disclosed. Prostate-specific membrane antigen (PSMA) is an important biomarker that can bond to the surface of prostate cancer cells with levels proportional to tumor grade. PSMA can bond to ICN, and can travel through vasculature to the prostate. At the prostate, the PSMA-ICN compound binds to the cancer cells, and during the chemical bonding process, the ICN is liberated and becomes a somewhat “free” molecule. The ICN stays in the region of the prostate for about 19 days. The combination of free ICN (liberated from the PSMA-ICN molecule), once insonated by the energy from the phased array Doppler or the photoacoustic device, would allow the user to locate potential cancer sites and aid in the early detection of many cancers because the ICN molecule has a different absorption spectra than any free PSMA-ICN or other photo-acoustic responsive molecules nearby. That is, by sweeping the frequency of the input light, a different photo-acoustic response will arise from each molecular species, hence differentiating them in vivo can occur.

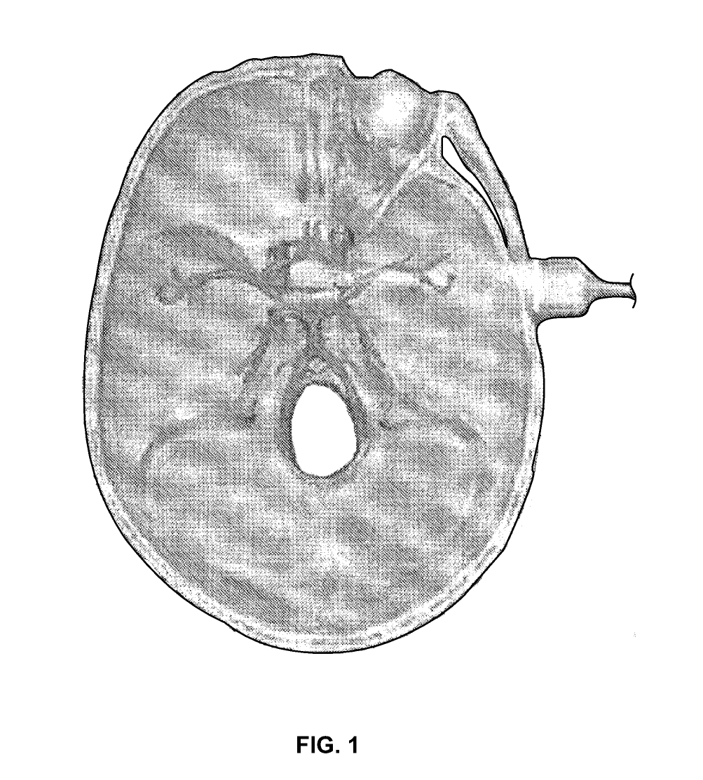

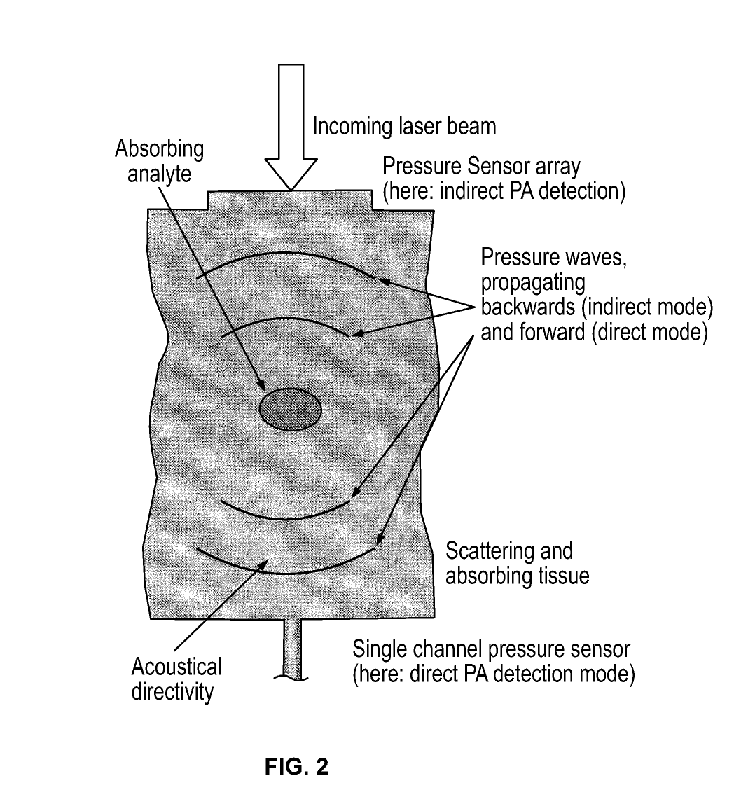

FIG. 1 and FIG. 2 generally illustrate approaches for using advanced imaging to ascertain, for example, the status of ostensively deleterious brain-challenging events in process. In FIG. 1, a diagram of cerebral vasculature in the skull is shown, obtained using a transcranial Doppler probe. This figure presents a typical transcranial Doppler in the pre-temporal skull region to evaluate the anterior circulation including but not limited to the middle cerebral arteries and anterior cerebral arteries. Probe placement for the ophthalmic artery eye paste on probes and back of the head occipital probes are not shown. The light is brought to the skull surface first in a fiber optic cable, and then diffused with a field lens, and then mounted with a flexible mount to contact the skull, possibly in a area of the skull devoid of hair. As described earlier, typical wavelengths of light used can penetrate (with substantial attenuation but still strong photo-acoustic signal) easily 4 cm into the skull. Similar probes would be used to insonate the left anterior circulation and also at the back of occiput for the basilar artery and vertebral artery for transcranial Doppler and other examinations (not shown). Ultrasound probes would also be used to insonate the neck vessels, including bilateral carotid and vertebral arteries for the carotid Doppler examination. Probes would be affixed with in the helmet as shown in the subsequent figures. The probe placement could also be used for optoacoustic and phased array probes.

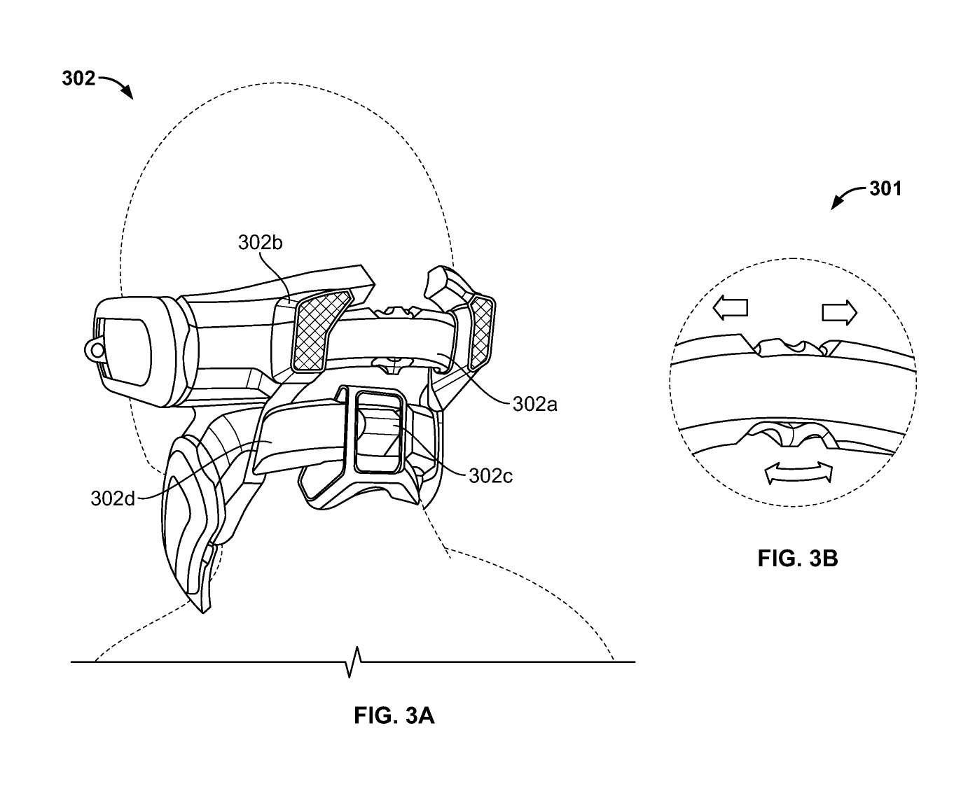

FIG. 2 specifically outlines the methods for optoacoustic spectroscopy. A diagram of the photo-acoustic laser beam source, with consequent sound waves being produced due to photoacoustic effect. Analyte in this case could be brain vasculature, or more importantly an area where an artery has hemorrhaged, and substantial blood is present outside of the vasculature. Such blood would not have any velocities but would be have very small Doppler signal. The photo-acoustic probes and system would be used to look at brain oxygenation and cerebral perfusion in combination with transcranial Doppler and ICN green dye. Referring now to FIGS. 3 through 6, illustrations of apparatus for arraying, moving and positioning imaging equipment on a patient, for example, in an ambulance that may be having a stroke, is disclosed.

FIG. 3 demonstrates how, for example, ultrasound transducers can bracingly engage without restricting a user's head. A diagram of the patient mountable TCD (or Photo-acoustic) ultrasound pick-ups device, with adjustment points shown and degrees of freedom so device can be mounted on patient and adjusted to fit any size neck or head. FIG. 3 schematically illustrates Novel Enhanced Apparatus with Helmet/Carotid Collar Means On-Boarded. As shown in FIG. 3, the helmet size can be adjusted by the adjustment controls 301 to accommodate patient variance in head size. As shown at FIG. 3, 302 indicates a posterior view of the head support structure, that has a size adjustment dial 302a, non-slip supports 302b, a thoracic sensor 302c, and a sensor cartridge holder 302d on a positional rail.

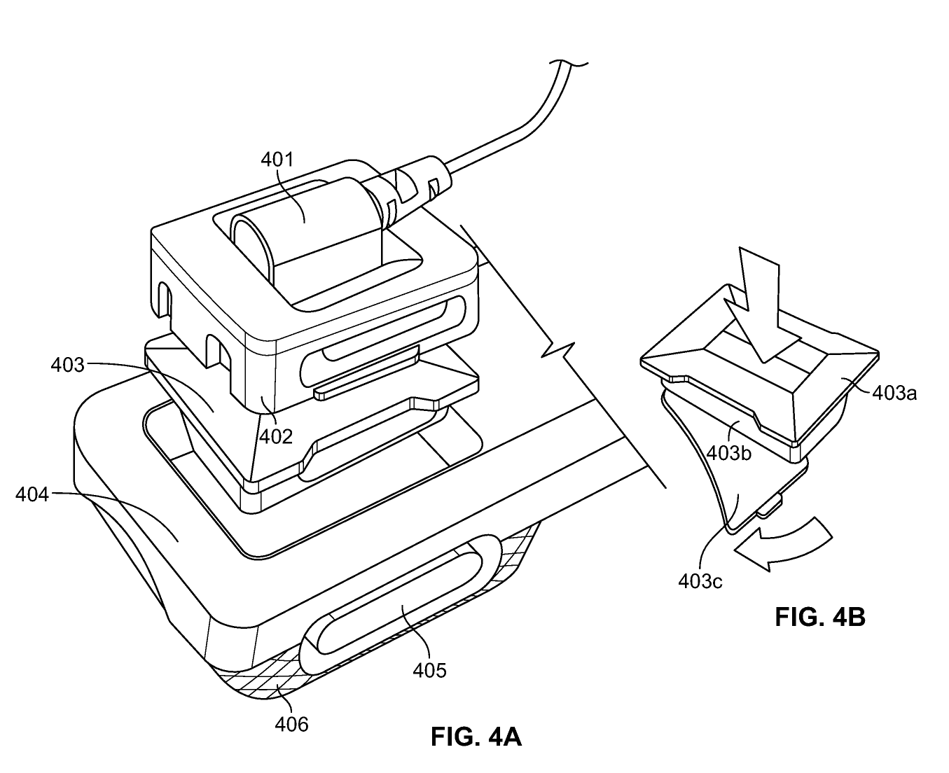

Turning now also to FIG. 4, modular connections allow the flow of data in real-time and interface with other aspects of, for example, stroke pre hospital care and telemedicine. FIG. 4 further illustrates the Novel Enhanced Apparatus with Helmet/Carotid Collar Means On-Boarded. FIG. 4 further illustrates a diagram of the transcranial Doppler (TCD) detector (top of the stack), and one embodiment of motor-actuators for positioning the TCD detector for optimal signal strength. The stack of positioners allows movement in x, y, and angles so that the TCD or photo acoustic ultrasound signal can be optimized. FIG. 4 shows the modular sensor cartridge assembly and positioning arm. The modular sensor cartridge assembly and positioning arm comprises a sensor module 401, also referred to herein as a probe; a sensor control cartridge 402; a disposable ultrasound gel pack 403; a sterile perforated panel for sensor cartridge to pass through 403a; an ultrasound gel compartment 403b; a pull away sterile cover for patient contact side of cartridge 403c; a positioning arm 404; a cartridge release button 405; and an underside antibacterial fabric covered padding 406. It should be appreciated that the sensor module may be a carotid Doppler transducer, a transcranial Doppler transducer, a transducer, a photoacoustic probe, an optoacoustic tomography probe, or a phased array, as discussed herein. Phased array and photoacoustic can be integrated into the helmet at similar positions or contiguous positions.

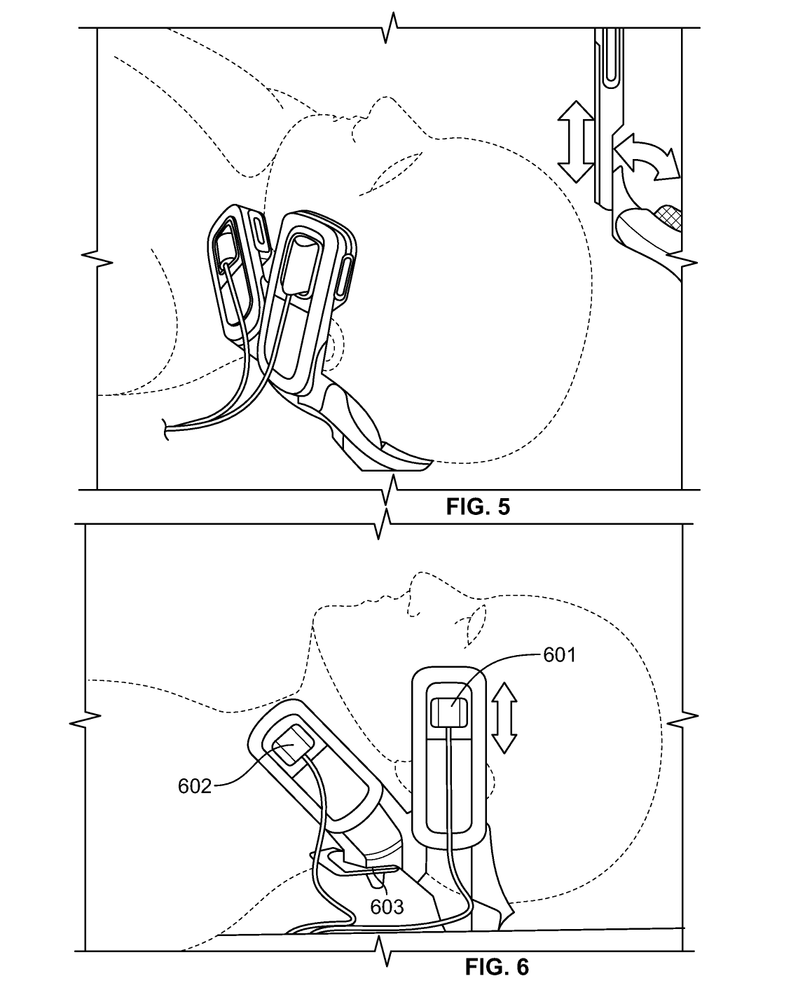

FIGS. 5 and 6 show how positioning of carotid and brain arteries can be done according to these inventions. FIG. 5 further illustrates Novel Enhanced Apparatus with Helmet/Carotid Collar Means On-Boarded. A diagram of the x-y actuators positioning scheme is shown. The left panel illustrates sensor cartridges installed and positioned in both helmet and collar positioning arms. The right panel illustrates how the positioning arm will move transversely and rotate along axes perpendicular to transverse movement. Positioning arm width angle adjusts to accommodate a range of patient head size and anthropomorphics.

FIG. 6 provides a profile view of helmet and collar apparatus also illustrating transverse positional adjustment of helmet senor and positioning arm. A diagram of how the two degrees of freedom are attained by a rotation about the bottom of the sensor holder, and then a translation along the axis (arrow shown) of the sensor. This allows virtually any positioning point to be obtained more easily with simpler parts than a pure x-y rotation stage. The device as shown in FIG. 6 comprises a temporal sensor 601, a carotid sensor 602, and a cervical sensor 603.

For FIGS. 3, 4, 5, and 6, the exterior shell of the helmet is made of ABS/Polycarbonate. Interior form of the helmet is EPS foam (expended polystyrene). Mechanical components are ABS and metal. Padded surfaces that come in contact with the patient would be EVA foam. The gel cartridge has a Polypropylene cover and Polyethylene gel holder peal away bottom cover (Velaron).

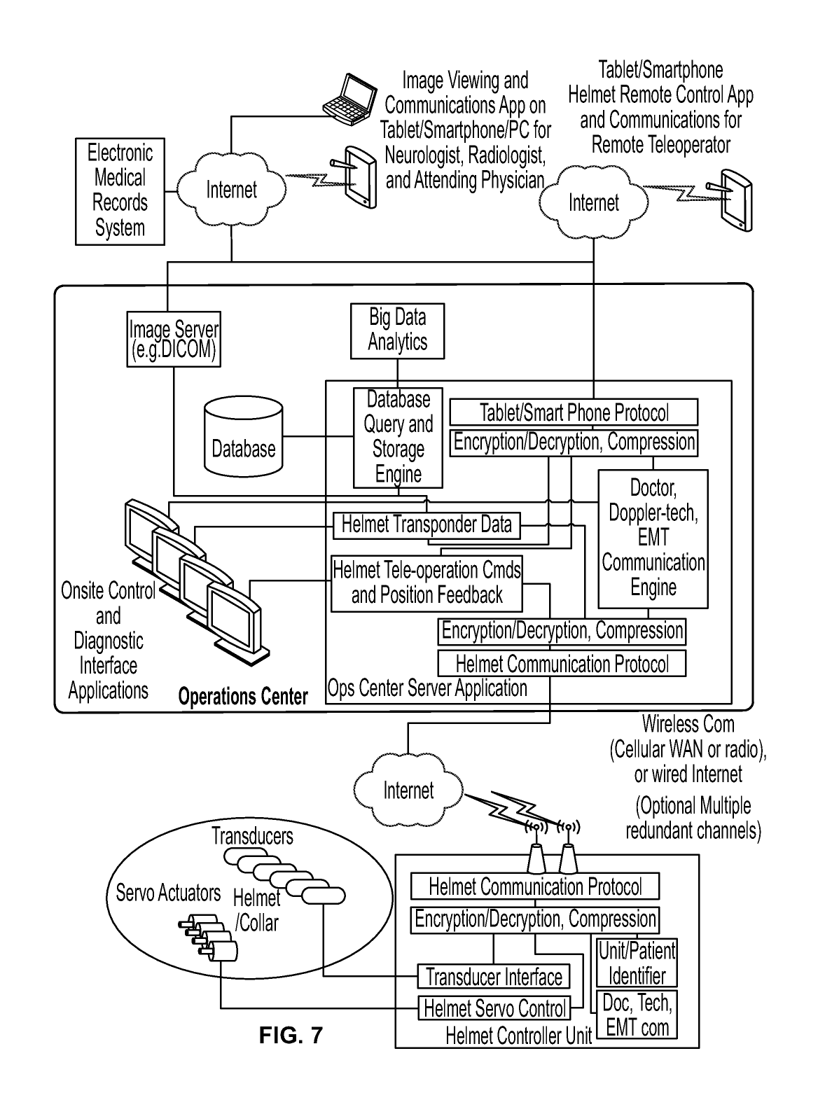

Likewise, and further including FIG. 7, those skilled in the art will understand how the data-flow is managed according to the instant teachings. FIG. 7 illustrates the major components and communications pathways for the deployed helmet/collar system.

The bottom of the figure shows Helmet/Collar Apparatus (hereafter H/CA) containing the ultrasound transducers and the servo actuators for positioning the transducers. The helmet/collar system is connected to a Helmet Controller Unit (hereafter HCU) via power and data cabling. The HCU resides in physical proximity to the H/CA at the remote location where the patient is. The HCU provides communications and control interfaces between the Operations Center, the onsite medical technician operating the H/CA, and the H/CA hardware. In practice the HCU may consist of one or more physical packages that contain a portable hardened PC or equivalent computing platform with a display, audio and video communications capability, encryption, data compression, a keyboard for data entry, and a wireless or wired internet connectivity capability. In the case of a wireless system, there may be multiple transmit and receive units operating on different frequencies or utilizing different cell phone carriers for data redundancy.

The Operations Center provides a nexus for communication with multiple remotely located H/CAs. Data is processed through a central system of servers running an application that provides voice and video, textual data, imaging data, telemetry and tele-operation command communications. The data is routed to an available Operations Center specialist that is trained to operate the tele-operations and has radiology expertise to acquire usable image and Doppler data. The Operations Center may consist of pool of radiology tele-operations specialists available to handle data from multiple patients simultaneously. All data surrounding an application of the H/CA is collected in a data base (e.g. time, date, patient id, ambulance, location, images, H/CA telemetry, radiologist or neurologist ids, emergency room attending physician ids, operations specialist ids). The database provides internal records for traceability as well as the data to be accessed as part of big data analytics. The Operations Center specialist will establish communications connections with a qualified diagnostician (e.g. neurologist or radiologist) who will perform the actual assessment of the patient and will provide the consultation to the attending physician. Once the tele-operation specialist has acquired usable images, the images will be uploaded to a medical image server where they will be dispatched to the diagnostician and the attending physician, at which time the images also become part of the patient's electronic medical records.

Diagnosticians (radiologists, neurologists) may be at the Operations Center or remote, as illustrated in the figure. An additional remote application can also allow for remote tele-operators. In this way an external pool of additional diagnosticians and tele-operators may be on call as load demands. In addition to the command and telemetry interface for controlling the H/CA, the remote tele-operator will also have full communications with all diagnosticians, physicians, and ambulance personnel involved in the patient that is assigned to them by the Operations Center.

While methods, devices, compositions, and the like, have been described in terms of what are presently considered to be the most practical and preferred implementations, it is to be understood that the disclosure need not be limited to the disclosed implementations. It is intended to cover various modifications and similar arrangements included within the spirit and scope of the claims, the scope of which should be accorded the broadest interpretation so as to encompass all such modifications and similar structures. The present disclosure includes any and all implementations of the following claims. It is understood that the term, present disclosure, in the context of a description of a component, characteristic, or step, of one particular embodiment of the disclosure, does not imply or mean that all embodiments of the disclosure comprise that particular component, characteristic, or step. It should also be understood that a variety of changes may be made without departing from the essence of the disclosure. Such changes are also implicitly included in the description. They still fall within the scope of this disclosure. It should be understood that this disclosure is intended to yield a patent covering numerous aspects of the disclosure both independently and as an overall system and in both method and apparatus modes.

Further, each of the various elements of the disclosure and claims may also be achieved in a variety of manners. This disclosure should be understood to encompass each such variation, be it a variation of an implementation of any apparatus implementation, a method or process implementation, or even merely a variation of any element of these.

Particularly, it should be understood that as the disclosure relates to elements of the disclosure, the words for each element may be expressed by equivalent apparatus terms or method terms—even if only the function or result is the same.

Such equivalent, broader, or even more generic terms should be considered to be encompassed in the description of each element or action. Such terms can be substituted where desired to make explicit the implicitly broad coverage to which this disclosure is entitled.

It should be understood that all actions may be expressed as a means for taking that action or as an element which causes that action.

Similarly, each physical element disclosed should be understood to encompass a disclosure of the action which that physical element facilitates.

Any patents, publications, or other references mentioned in this application for patent are hereby incorporated by reference.

In this regard it should be understood that for practical reasons and so as to avoid adding potentially hundreds of claims, the applicant has presented claims with initial dependencies only. Support should be understood to exist to the degree required under new matter laws—including but not limited to United States Patent Law 35 USC §132 or other such laws—to permit the addition of any of the various dependencies or other elements presented under one independent claim or concept as dependencies or elements under any other independent claim or concept. To the extent that insubstantial substitutes are made, to the extent that the applicant did not in fact draft any claim so as to literally encompass any particular implementation, and to the extent otherwise applicable, the applicant should not be understood to have in any way intended to or actually relinquished such coverage as the applicant simply may not have been able to anticipate all eventualities; one skilled in the art, should not be reasonably expected to have drafted a claim that would have literally encompassed such alternative implementations.

Further, the use of the transitional phrase “comprising” is used to maintain the “open-end” claims herein, according to traditional claim interpretation. Thus, unless the context requires otherwise, it should be understood that the term “compromise” or variations such as “comprises” or “comprising”, are intended to imply the inclusion of a stated element or step or group of elements or steps but not the exclusion of any other element or step or group of elements or steps. Such terms should be interpreted in their most expansive forms so as to afford the applicant the broadest coverage legally permissible.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

1. A device for determining brain and neck arterial blood vessel anatomy, blood flow velocity, presence of arterial stenosis or obstruction, and determination of brain oxygenation comprising, in combination:
an array of ultrasound transducers;
actuators coupled to the array of ultrasound transducers;
said actuators enabled to alter, skew, move, rotate, or change the position of the transducers;
said actuators enabled to be controlled remotely; and
the array of ultrasound transducers having the ability to receive and send Doppler shifted signals, regarding blood flow from brain and neck vasculatures and oxygenation, to a remote site.

2. The device according to claim 1, further comprising:
means for wireless remote control capability and remote manipulation of the ultrasound transducers; and
means for wireless transmission and receipt of the Doppler signals over internet, radio, land links, and related systems.

3. The device according to claim 1, further comprising means for making a 3-dimensional model of the blood flow of the brain, using at least a Super-resolution algorithm and angular positions from an ultrasound transducer encoder and a signal return time from a vasculature.

4. The device of claim 3, whereby spectral analysis of 3D information yields neck vessel Doppler velocities, resistances, pulse waveform anatomy, anatomy of the arteries, in addition to B mode gray scale imaging and color flow Doppler that yields information on internal carotid and other neck artery stenosis, obstruction, and plaque definition, localization, and extent.

5. A helmet and collar imaging system, the system comprising:
a helmet apparatus, wherein the helmet apparatus comprises:
head transducers; and
actuators engaged to the head transducers;
a collar apparatus, wherein the collar apparatus comprises:
collar transducers; and
actuators engaged to the collar transducers;
a controller unit in electrical communication with the helmet apparatus and the collar apparatus, wherein the controller unit comprises a computing platform.

6. The helmet and collar imaging system of claim 5, wherein the helmet apparatus and the collar apparatus are configured to integrate together to create a structurally rigid platform, wherein cables and a cable restraint are combined in association with the rigid member.

7. The helmet and collar imaging system of claim 5, wherein the head transducers and collar transducers are ultrasound transducers.

8. The helmet and collar imaging system of claim 7, wherein the collar ultrasound transducers send and receive energy to image in ultrasound B-mode imaging and color flow to acquire data for 2D slices or 3D reconstruction, wherein the images allow for examination of stenosis, occlusion, and pulse wave anatomy and artery anatomy in the arteries of the neck.

9. The helmet and collar imaging system of claim 7, wherein the collar ultrasound transducers send and receive energy to image in ultrasound B-mode imaging and color flow to acquire data for 2D slices or 3D reconstruction, wherein the images also include spectral images for pulse wave anatomy and blood flow velocity.

10. The helmet and collar imaging system of claim 7, wherein with the head ultrasound transducers, the head ultrasound transducers send and receive energy to acquire data to examine stenosis, occlusion, blood flow velocity, pulse wave anatomy, emboli, and collateral blood flow in the large and medium arteries of the brain.

11. The helmet and collar imaging system of claim 5, wherein the head and collar transducers are phased array transducers and are capable of acquiring data for 3D reconstruction, and are configured for obtaining data related to blood flow velocity, stenosis and occlusions.

12. The helmet and collar imaging system of claim 5, wherein the head and collar transducers are configured for phased array focused beam steering to obtain Doppler data, and are configured for obtaining data related to blood flow velocity, stenosis and occlusions.

13. The helmet and collar imaging system of claim 5, wherein the head transducers are photoacoustic spectroscopy transducers, wherein the photoacoustic spectroscopy transducers are configured to acquire oxygenation data at specific brain blood vessel territories.

14. The helmet and collar imaging system of claim 5, wherein the head transducers are transcranial Doppler transducers.

15. The helmet and collar imaging system of claim 5, wherein the collar transducers are carotid Doppler transducers.

16. The helmet and collar imaging system of claim 5, wherein the actuators of the collar assembly are configured to move and rotate the collar transducers to thereby scan the neck of a patient during ultrasound data acquisition; and wherein the actuators move the transducers along a linear track parallel or transverse to the major arteries of the neck.

17. The helmet and collar imaging system of claim 15, wherein the movement of the actuators is undertaken in discrete steps, with data acquired at each step in order to build a sampled series of image slices.

18. The helmet and collar imaging system of claim 15, wherein the movement of the actuators is in a continuous fashion with continuous sampling in order to build a high resolution 3-D data cube.

19. The helmet and collar imaging system of claim 5, wherein the actuators of the head assembly are configured to move and rotate in x, y, and z axis and thereby scan arteries of the anterior circulation, such as bilateral middle cerebral arteries, bilateral anterior cerebral arteries and posterior circulation, such as basilar artery and bilateral vertebral arteries.

20. The helmet and collar imaging system of claim 15, wherein the movement of the actuators is performed manually, by a mechanical spring or tension system, or by an electrical motor system.

21. The helmet and collar imaging system of claim 18, wherein the electrical motor system is controlled remotely or by controlled by a computer program.

22. The helmet and collar imaging system of claim 5, wherein the computing platform sends data acquired by the head and collar transducers to a remote computer.

23. The helmet and collar imaging system of claim 8, whereby the data acquired within specific Doppler gates used to build the color Doppler image for carotid and other neck arteries, are sent remotely.

24. The helmet and collar imaging system of claim 23, wherein sampling up to every point of the color Doppler or power Doppler, to measure the velocity and other parameters by 3D reconstruction from 2D slices or 2D alone using single-crystal probes, linear arrays, or 2-D phased arrays.

25. The helmet and collar imaging system of claim 9, whereby the data acquired within specific Doppler gates used to build the power m mode display and spectral velocity display with wave anatomy, sent remotely and sampling relevant large and medium brain arteries at varying depths to measure the velocity and more advanced parameters.

26. The helmet and collar imaging system of claim 20, wherein the data acquired is processed to determine blood velocity, resistance index, and pulsatility of the vessels and arteries of the brain and blood velocity, b mode imaging, plaque characterization and extent, and color flow imaging in arteries of the neck, including but not limited to the internal carotid artery.

27. The helmet and collar imaging system of claim 20, wherein the acquired data is reconstructed into 3D images of the transcranial vessels.

28. The helmet and collar imaging system of claim 15, whereby movement of the actuators and transducers provides telemetry feedback information on its real-time position to a local or a remote operator.

29. The helmet and collar imaging system of claim 5, wherein the collar transducers are multi-crystal linear array transducers, wherein the collar transducers are multi-crystal linear array transducers are aligned transverse or longitudinal to the major arteries to acquire images of bilateral carotid arteries and bilaterial vertebral arteries.

30. The helmet and collar imaging system of claim 15, wherein the collar transducers are rotated perpendicular to an artery to acquire data for generating a 3D reconstruction of the neck arteries, and wherein the collar transducers are positioned parallel to an artery to acquire higher resolution Doppler data.

31. The helmet and collar imaging system of claim 5, wherein the collar and head transducers are removable from the actuators.

32. The helmet and collar imaging system of claim 5, further comprising a modular unit that is removeably coupled to the helmet apparatus.

33. The helmet and collar imaging system of claim 5, further comprising impedance matching inserts that are contained in a cartridge; wherein, the cartridge is removeably coupled to the helmet apparatus.

34. The helmet and collar imaging system of claim 20, wherein the acquired data is compared to a database that holds matrices of healthy or sick patients to help diagnose or indicate risk zones, and wherein the data is sent to a remote data center for finding a best fit to existing internal carotid and other neck vessel Doppler and Color-Doppler and B-Mode images, phased array, transcranial doppler artery data, and photoacoustic spectroscopy data to existing patient archival data for further understanding of vasculature and blood flow and oxygenation.

35. The helmet and collar imaging system of claim 15, further comprising a sensor, wherein the sensor is a continuous wave/pulsed wave sensor positioned proximate to a head or collar transducer, wherein the transducer is an ultrasound transducer, used for optimal alignment to a vessel by chirping, further comprising a feedback system, either an audio tone mechanism or a visual system.

36. The helmet and collar imaging system of claim 35, wherein the head transducers are able to acquire measurements to find the acoustical window through the bone by the measurement of the impedance, preferably by raw data transfer and radiofrequency (RF) analyses.

37. The helmet and collar imaging system of claim 36, wherein the acoustical impedance is matched, and the transcranial settings are changed for better penetration.

38. The helmet and collar imaging system of claim 37, wherein frequencies are adjusted frequencies for each transducer based upon the bone medium the energy from the transducers are impeded by.

39. The helmet and collar imaging system of claim 5, wherein the head and collar transducers receive and transmit energy, wherein transmitting and receiving systems employ lossless data compression, data encryption, or error detection and correction encoding for transmitted commands and data; and wherein the system connects to a data system through wireless channels, wherein the use of multiple wireless channels simultaneously reduces dropout in the moving ambulance, and packet tracking to discard duplicates and to detect missing packets due to dropout.

40. The helmet and collar imaging system of claim 39, further comprising a receiver for handling communications between the helmet and collar imaging system and a centralized operations center system, wherein the data from the helmet and collar imaging system is simultaneously communicated to the centralized operations center system, where the tele-operation control is dispatched to an available operator and the acquired Doppler and image data is dispatched to a remote medical specialist for analysis; wherein the data is converted into images used in the analysis are then transmitted to the attending doctor and are logged electronically into the patient medical records; and wherein the system allows for two-way direct communications between tele-operators, the remote medical specialists, and the on-site medical technician or attending doctor.

41. The helmet and collar imaging system of claim 40, wherein the centralized operations center system tracks metadata to relate the patient, the EMT, the tele-operator, the analyzing specialist (neurologist, radiologist, stroke neurologist), time and date, and location; and wherein the centralized system records tele-operation records of transducer positions where data is successfully acquired; and wherein the centralized operations center system monitors remote units for proper operation and flags units in need of field service or replacement.

42. The helmet and collar imaging system of claim 41, wherein the helmet and collar imaging system is placed on the head and neck region of a patient, and the scan is automated and is started by the EMT, and data is then sent to the data center without remote tele-operation; and wherein the scan is preprogrammed to sweep through a range of motion, or to use raw data feedback such as impedance and reflection monitoring in automatically in the controller unit in order to seek the optimal position for good signals, and wherein a rescan can be commanded by the remote tele-operator with modified parameters, or the tele-operator can take over manually.

43. The helmet and collar imaging system of claim 42, where manual operations may include direct control, commanded complex movements that are preprogrammed in the remote unit via firmware or software, and commands to go to specific positions based on telemetry data.

44. The helmet and collar imaging system of claim 43, further comprising marker stickers, wherein marker stickers are placed on the patient's skin to allow the helmet, including head and collar, to maintain a fixed reference on the patient through an optical means; wherein a high response servo system can then track the fixed reference to correct for any movement between the helmet and the transducer, and maintain the relative position in order to keep the data acquisition optimized even while the vehicle is bouncing around; and as an alternative to high response servo, the analysis system can monitor the motion and correct in software image processing when possible based on the known motion, or drop or otherwise qualify records when the data is acquired from too far out of position.

45. The helmet and collar imaging system of claim 12, further comprising administrating to a patient ICN green or other photo-acoustic or fluorescing agent and using the photoaccoustic transducers to detect presence of the ICN green.

46. The helmet and collar imaging system of claim 45, wherein ICN green is detected by a Doppler shifted acoustic spectrum caused by the motion shift of the ICN in the vasculature.

47. The helmet and collar imaging system of claim 45, wherein the photoaccoustic transducers detect and differentiate hemoglobin and deoxyhemoglobin to determine viable vasculature.

48. The helmet and collar imaging system of claim 47, photoaccoustic spectroscopy with ICN, combined with phased array or transcranial Doppler is used to determine cerebral blood volume, cerebral blood flow and mean transit time to determine cerebral perfusion in completed stroke, and penumbral and normal tissue.

49. A method for physiological assessment of brain artery openings, closures or contiguous tissue efficacy in conjunction with brain intra-arterial clot buster or clot removal by catheter based devices, the method comprising:
providing a catheter defining a lumen, wherein the lumen contains an intravascular multi-headed probe, wherein the catheter is introduced into a patient and threaded up to a cerebral host artery of interest; and
evaluating the efficacy of a procedure by acquiring data through the intravascular multi-headed probe simultaneously or serially pre intervention, during intervention, and after intervention.

50. The method of claim 49, wherein the multi-headed probe comprises small ultrasound transducers, including transcranial Doppler transducer and phased array transducers, as well as photoacoustic spectroscopy transducers.

51. The method of claim 50, wherein the ultrasound transducers image and evaluate blood flow, stenosis and occlusion in the catherterized artery and contiguous medium and large arteries, including other arteries besides the host artery.

52. The method of claim 50, wherein the optoacoustic probe evaluates tissue oxygenation at the arterial stroke site and contiguous brain sites for stroke, pre therapy, during and after therapy with intra-arterial clot buster or clot remover.

53. The method of claim 50, wherein the multi-head probe determines tissue efficacy and injury pre and post intra-arterial substance injection, including but not limited to tpa or other clot busters.

54. The method of claim 50, wherein the data from multi-head probe analysis pre, during, and post catheter based therapy assists in treatment decisions and treatment efficacy in combination with standard contrast based imaging of arteries at stroke sites.

55. The method of claim 51, wherein the images are sent for remote analysis to an operations center,

56. The method of claim 51, wherein images are acquired at every nth frame.

57. The helmet and collar imaging system of claim 11, further comprising an element spacing in the array that is greater than, equal to or less than a half wavelength of the ultrasound energy produced by the elements, and wherein the array is used differently in transmit and receive modes, further comprising:
forming a transmit beam from a position external to a region of interest encompassing a plurality of receive beams and initially acquiring a signal by insonating a target region comprising multiple receive beam positions over a substantially simultaneous period;
receiving data from the multiple receive beam positions of the array;
combining the received data in a processor; and
locking onto the receive beam and the point(s) producing a peak signal; and correcting for motions in the target region by periodically forming multiple receive beams and re-acquiring the peak signal.

58. The helmet and collar imaging system of claim 5, further comprising a data reduction and analysis system wherein actuators coupled to ultrasound transducers can be remotely manipulated, over the Internet or radio or land links, with control taking place at a remote site distal from the patient.

59. The helmet and collar imaging system of claim 5, wherein said actuators may comprise robotic arms or other robotic manipulation systems that enable a transcranial doppler (TCD) probe or phased array probe to move in space, approach and make gentle contact with the patient's head, and begin searching for arterial Doppler signals.

60. The helmet and collar imaging system of claim 5 further comprising a means to:
make maps of brain vasculatures;
identify acute occlusion or stenosis in major brain and neck arteries;
remotely send the data identified, to a remote site; and
provide capabilities to quickly analyze the data identified and advise delivery of the patient to a primary or comprehensive stroke center upon finding an occlusion or stenosis in major brain or neck arteries.
